(19) United States
(12) Patent Application Publication
(10) Pub. No.: US 2009/0220552 A1
(43) Pub. Date: Sep. 3, 2009

(54) FORMULATIONS CONTAINING PANTOPRAZOLE FREE ACID AND ITS SALTS

(75) Inventor: Sergio Lloret Perez, Barcelona (ES)

Correspondence Address: HOGAN & HARTSON LLP;IP GROUP, COLUMBIA SQUARE, 555 THIRTEENTH STREET, N.W., WASHINGTON, DC, 20004, US

(73) Assignee: COMBINO PHARM, S.L., Barcelona, ES (ES)

(21) Appl. No.: 11/914,334

(22) PCT Filed: Jun. 12, 2006
(86) PCT No.: PCT/IB06/03550

§ 371 Date: Jul. 8, 2008

Related U.S. Application Data
(60) Provisional application No. 60/680,528, filed on May 13, 2005.

(51) Int. Cl. A61K 9/00; A61K 31/4439

(52) U.S. Cl. 424/400; 514/338

(57) ABSTRACT

The invention relates to new formulations and dosage units of solid crystalline pantoprazole free acid and its salts (e.g., pantoprazole sodium sesquihydrate) that are resistant to gastric juice digestion and are useful for the therapeutic treatment (including prophylactic treatment) of mammals, including humans, and a process for making the same

### CROSS REFERENCE TO RELATED APPLICATIONS

This application claims priority to U.S. Provisional Application No. 60/680,528, filed May 13, 2005, which application is expressly incorporated herein by reference in its entirety.

### BACKGROUND OF THE INVENTION

1. Field of the Invention

The invention relates, in general, to new formulations and dosage units of solid crystalline pantoprazole free acid and its salts (e.g., pantoprazole sodium sesquihydrate) that are resistant to gastric juice digestion and are useful for the therapeutic treatment (including prophylactic treatment) of mammals, including humans, and a process for making the same.

2. Relevant Background

Pantoprazole (5-(difluoromeoxy)-2-[[(3,4-dimethoxy-2-pyridinyl)methyl]sulfinyl]-1H-benzimidazole) is a benzimidazole compound that inhibits gastric acid secretion. Pantoprazole sodium sesquihydrate has been approved by the FDA for parenteral administration under the name Protonix IV® and for oral administration under the name Protonix®, for short-term treatment of erosive esophagitis associated with gastroesophageal reflux disease (GERD), for maintenance of healing of erosive esophagitis and for the treatment of pathological hypersecretory conditions, including, for example, Zollinger-Ellison syndrome.

The pharmaceutically active ingredient pantoprazole is disclosed U.S. Pat. No. 4,758,579 (equivalent to EP 0 166 287), which characterizes pantoprazole only by its melting point.

Protonix is marketed in the form of a delayed release tablet, which is resistant to gastric juice, and consists of: (i) a core, (ii) an outer layer resistant to gastric juice layer and (iii) an inert intermediate layer between the core and outer layer, which are not compatible with one another, in order to protect the active ingredient from the outer layer.

U.S. Pat. No. 5,997,903 (equivalent to EP 589 981 B) discloses an orally administrable medicament in pellet or tablet form that is resistant to gastric juice which consists of (i) a core of active compound (or its physiologically-tolerated salt) admixed with binder, a filler and, optionally, another tablet auxiliary or basic physiologically-tolerated inorganic compound, (ii) an inert water-soluble intermediate layer surrounding the core and (iii) an outer layer which is resistant to gastric juice. The active compound is pantoprazole, the binder is polyvinylpyrrolidone and/or hydroxypropylmethylcellulose and, optionally, the filler is mannitol.

Thus, according to U.S. Pat. No. 5,997,903, oral pharmaceutical compositions of pantoprazole are described that do not create problems of stability of the active ingredient by using a selected binder and filler in the core. The binder materials described therein are polyvinylpyrrolidone and/or hydroxypropylmethylcellulose and are combined with mannitol as the inert filler to minimize instability of the active ingredient. According to U.S. Pat. No. 5,997,903, therefore, mannitol cannot be used as the sole or only filler for pantoprazole tablets absent the inclusion of a suitable binder capable of imparting an adequate hardness to the core.

Furthermore, commercially-marketed Protonix tablets contain sodium carbonate in the core as a basic physiologically-tolerated inorganic compound. The use of a carbonate salt can cause handling difficulties during processing because part of the carbonate salt can be hydrolyzed by water or moisture to produce effervescence. Additionally, uniform distribution of the carbonate salt in the tablets is not consistently assured.

Thus, it is an objective of the invention to provide new oral pharmaceutical formulations or dosage units containing pantoprazole and/or its salts with improved stability and, in particular, with improved stability relative to such formulations and/or dosage units prepared using polyvinylpyrrolidone and/or hydroxypropylmethylcellulose as binders and/or sodium carbonate.

### SUMMARY OF THE INVENTION

The invention provides new formulations and dosage units of solid crystalline pantoprazole free acid and its salts (e.g., pantoprazole sodium sesquihydrate) that are resistant to gastric juice digestion and are useful for the therapeutic treatment (including prophylactic treatment) of mammals, including humans and a process for making the same.

### BRIEF DESCRIPTION OF THE DRAWING

The accompanying drawings, which are included to provide a further understanding of the invention and are incorporated in and constitute a part of this specification, illustrate embodiments of the invention and together with the description serve to explain the principles of the invention. In the drawings:

FIG. 1 illustrates the dissolution profile of a 40 mg formulation of pantoprazole obtained in Example 2 and the dissolution profile of a marketed formulation (40 mg tablet) of pantoprazole (i.e., Protonix®).

### DETAILED DESCRIPTION OF THE PREFERRED EMBODIMENTS

Reference will now be made in detail to the preferred embodiments of the invention.

This invention may, however, be embodied in many different forms and should not be construed as limited to the embodiments set forth herein. In addition, and as will be appreciated by one of skill in the art, the invention may be embodied as a method, system or process.

The invention provides new formulations and dosage units of solid crystalline pantoprazole free acid and its salts (e.g., pantoprazole sodium sesquihydrate) that are resistant to gastric juice digestion and are useful for the therapeutic treatment (including prophylactic treatment) of mammals, including humans and a process for making the same.

In particular, the formulations of the invention can be formulated as stable solid oral forms in the presence of mannitol, acting as a filler and as a binder. In addition to mannitol, other excipient materials, including lubricants (e.g., calcium salts of higher fatty acids), release agents and tablet disintegrating agents (e.g., croscarmellose sodium) can also be included in the formulations.

The formulations and/or dosage units of the invention include a quantity of pantoprazole and/or its salts (e.g., 20 mg and 40 mg) and include (i) a core that includes, among other things, the active ingredient mixed with an alkaline reacting compound (e.g., trisodium phosphate) and mannitol, (ii) an inert and insulating intermediate layer surrounding the core, and (iii) an outer layer (i.e., the enteric layer) resistant to gastric juice.

In the formulations, the amount of mannitol is approximately 0.6 to approximately 4 parts (by weight), and more preferably approximately 1.8 to approximately 2.7 parts (by weight), per part (by weight) of the pantoprazole sodium sesquihydrate used in the formulations.

In the formulations, mannitol powder and spray dried mannitol are used.

In the formulations, the amount of mannitol powder is approximately 0.5 to approximately 3 parts (by weight), and more preferably approximately 1.5 to approximately 2 parts (by weight), per part (by weight) of the pantoprazole sodium sesquihydrate used in the formulations. Similarly, the amount of spray dried mannitol is approximately 0.1 to approximately 1 parts (by weight), and more preferably approximately 0.3 to approximately 0.7 parts (by weight), per part (by weight) of the pantoprazole sodium sesquihydrate.

As described below, the tablet-core may be prepared in a conventional manner. For example, a core in tablet form is obtained by mixing pantoprazole sodium sesquihydrate with mannitol powder, croscarmellose and an aqueous solution of the alkaline reacting compound and granulating the obtained mixture and drying the granulate. The obtained granulate is mixed with spray dried mannitol, and optionally the lubricant, and thereafter tableted.

In another aspect of the invention, the formation of the intermediate coating layer by coating of the core is performed until a specific weight is achieved. Namely, the specific weight to be obtained is a 15% weight increase of the uncoated tablets for 40 mg tablet formulations and a 19% weight increase of the uncoated tablets for the 20 mg tablet formulations.

In another aspect of the invention, once the core is coated with the intermediate coating layer, it is further treated with an enteric coating to provide a stabilized formulation of an otherwise acid-unstable compound. In this regard, the formation of the enteric coating layer be carried out in a conventional manner until a specific weight is achieved. Namely the specific weight to be obtained is a 9% weight increase for the 40 mg tablets and a 11% weight increase for the 20 mg tablets.

The core includes the active ingredient in the form of pantoprazole and/or its salts, including in particular, pantoprazole sodium sesquihydrate, as well as additional excipient materials. The excipient materials can include, but are not limited to, filler and/or binder materials (e.g., mannitol), disintegrant materials (e.g., croscarmellose sodium); lubricant materials (e.g., calcium stearate); and pH regulators (e.g., trisodium phosphate or a hydrate thereof). A representative filler and/or binder material suitable for use in the invention is spray dried mannitol (e.g. Pearlitol SD 200®), which is a mannitol useful in direct compression.

Trisodium phosphate or hydrate thereof (e.g., 0.8 mg of Na₃PO₄.H₂O per 20 mg of active ingredient) can be used in the core to regulate the pH. Trisodium phosphate monohydrate is a buffer of alkaline zone that, when partially hydrolyzed, gives rise to the system Na₃PO₄/Na₂PO₄ but without effervescence.

The insulating intermediate layer includes a polymer (e.g., hydroxypropylmethylcellulose (HPMC), which is commercially available as, for example, Methocel E3LV®) and plasticizers (e.g., polyvinylpyrrolidone (also called povidone, which is commercially available as PVP) and propylene glycol).

The outer enteric layer includes a copolymer of methacrylate/acrylic acid (commercially available as Eudragit L®), a plasticizer (e.g., triethyl citrate) and dyes.

The invention also comprises a process for preparing the tablets of the present invention.

The process includes a granulation process, an intermediate finishing process, a compression step, a first coating step (i.e., insulating step) and a second coating step (i.e., enteric coating step).

Granulation Step

Suitable quantities of mannitol powder, sodium pantoprazole sesquihydrate and croscamellose sodium are first weighed and sieved and then combined in a high shear granulator. Sodium phosphate 12-hydrate in also dissolved in deionized water. Next, the ground mixture of mannitol powder, sodium pantoprazole sesquihydrate and croscamellose sodium is combined with the sodium phosphate solution (i.e., an aqueous solution of the alkaline reacting compound), and the combined mixture is calibrated by passing it through an appropriate sieve and then granulated. The combined mixture is then dried in a fluid bed for approximately 1 hour at approximately 30° C. until a water content of less than 3.5% (Karl Fischer) or 2% loss on drying is achieved. The dried mixture is then sieved.

Intermediate Finishing Step

The product obtained in the granulation step is weighed, sieved and mixed. It is then combined and mixed with spray dried mannitol (e.g., Pearlitol®) in a container blender for approximately 15 minutes. Calcium stearate is then calibrated by passing it through a sieve and combining it with the previous mixture for approximately one minute.

Compression Step

The mixture from the intermediate finishing step is then compressed under suitable conditions to produce cores. The pressed cores are stored in a dry place in a double bag and silica gel and protected from light.

The resulting tablet-cores have adequate hardness and low friability which are suitable for coating without chipping or breaking problems. Namely, the tablet-cores produced have a hardness of approximately 40N to approximately 60N, and a friability of approximately 0.1% to approximately 0.7%.

First Coating Step (i.e., Insulating Step)

Propylene glycol is dissolved in deionized water. Polyvinylpyrrolidone .e.g. PVP K25®) and hydroxypropylmethylcellulose .e.g. Methocel E3LV®) are added to and dissolved in the propylene glycol solution. The solution is then coated on the previously prepared cores until the desired weight increase is achieved and allowed to dry.

Second Coating Step (i.e., Enteric Coating Step)

Triethyl citrate, yellow ferric oxide A and titanium dioxide are gently mixed in deionized water and calibrated. The solution is then added to an aqueous dispersion of ethyl acrylate-methacrylic acid copolymer (1:1).e.g. Eudragit L30D-55®) and stirred. The solution is then coated over the tablets obtained in the first coating step (i.e., insulating step) until the desired weight increase is achieved and allowed to dry.

Although the invention has been described and illustrated with a certain degree of particularity, it is understood that the present disclosure has been made only by way of example, and that numerous changes in the conditions and order of steps can be resorted to by those skilled in the art without departing from the spirit and scope of the invention.

It is believed that this new formulation of pantoprazole and/or its salts is bioequivalent to Protonix®.

It will be apparent to those skilled in the art that various modifications and variations can be made in the present invention and specific examples provided herein without departing from the spirit or scope of the invention. Thus, it is intended that the present invention covers the modifications and variations of this invention that come within the scope of any claims and their equivalents.

The following examples are for illustrative purposes only and are not intended, nor should they be interpreted to, limit the scope of the invention.

### EXAMPLE 1

### Formulations of Pantoprazole Sodium Sesquihydrate

Table 1 illustrates formulations of pantoprazole sodium sesquihydrate at various concentrations of active ingredient.

TABLE 1

| | 20 MG | 40 MG |
|---|---|---|
| | TABLET | TABLET |
| COMPONENT | (MG) | (MG) |
| CORE | | |
| Pantoprazole Sodium Sesquihydrate¹ | 22.550 | 45.1 |
| Mannitol | 39.675 | 79.35 |
| Trisodium Phosphate Monohydrate² | 0.800 | 1.6 |
| Croscarmellose Sodium | 0.775 | 1.55 |
| Pearlitol SD200 ® | 12.195 | 24.39 |
| Calcium Stereate | 1.505 | 3.01 |
| INSULATING INTERMEDIATE LAYER | | |
| Deionized Water | — | — |
| Methocel E3LV ® | 11.88 | 19.00 |
| (Hydroxypropylmetilcelulose) | | |
| PVP K25 ® | 0.24 | 0.38 |
| Propylene glycol | 2.66 | 4.25 |
| OUTER ENTERIC LAYER | | |
| Deionized Water | — | — |
| Eudragit L ®³ | 7.94 | 14.13 |
| Triethyl Citrate | 0.82 | 1.45 |
| Yellow Ferric Oxide A | 0.02 | 0.03 |
| Titanium Dioxide | 0.21 | 0.34 |
| TOTAL | 101.27 | 194.58 |

Table 1 Notes:
¹22.55 mg of sodium pantoprazole sesquihydrate is equivalent to 20 mg of pantoprazole free acid, while 45.1 mg of sodium pantoprazole sesquihydrate is equivalent to 40 mg of pantoprazole free acid.
²Trisodium phosphate monohydrate is added as sodium phosphate 12-hydrate and 0.8 mg of trisodium phosphate monohydrate is equivalent to 1.67 mg of sodium phosphate 12-hydrate while 1.6 mg of trisodium phosphate monohydrate is equivalent to 3.34 mg of sodium phosphate 12-hydrate.
³Eudragit L ® is a copolymer of methacrylate/acrylic acid.
⁴Water is eliminated from the formulations in a drying step during processing.

### EXAMPLE 2

### Formulations of Pantoprazole Sodium Sesquihydrate

Table 2 illustrates formulations of pantoprazole sodium sesquihydrate at various concentrations of active ingredient.

TABLE 2

| | 20 MG | 40 MG |
|---|---|---|
| | TABLET | TABLET |
| COMPONENT | (MG) | (MG) |
| CORE | | |
| Pantoprazole Sodium Sesquihydrate¹ | 22.550 | 45.10 |
| Mannitol Powder | 39.755 | 79.51 |
| Trisodium Phosphate² | 0.720 | 1.44 |
| (added as Trisodium Phosphate 12-hdte) | | |
| Purified Water (1)³ | — | — |
| Croscarmellose Sodium | 0.775 | 1.55 |
| Spray Dried Mannitol | 12.195 | 24.39 |
| Calcium Stearate | 1.505 | 3.01 |
| TOTAL CORE | 77.500 | 155.00 |
| INSULATING INTERMEDIATE LAYER | | |
| Purified Water (2)³ | — | — |
| Hydroxypropylmethylcellulose | 11.88 | 19.00 |
| Povidone K25 ® | 0.24 | 0.38 |
| Propylene Glycol | 2.66 | 4.25 |
| TOTAL INSULATED CORE | 92.28 | 178.63 |
| OUTER ENTERIC LAYER | | |
| Purified Water (3)³ | — | — |
| Ethyl Acrylate-Methacrylic Acid Copolymer (1:1) | 7.94 | 14.13 |
| Sodium Laurylsulfate | 0.06 | 0.10 |
| Polysorbate 80 | 0.18 | 0.32 |
| Purified Water | 18.29 | 32.55 |
| (added as Eudragit L30-D55 ®⁴) | | |
| Triethyl Citrate | 0.82 | 1.45 |
| Iron Oxide “Yellow A” | 0.02 | 0.03 |
| Titanium Dioxide | 0.21 | 0.34 |
| TOTAL | 101.51 | 195.00 |

Table 2 Notes:
¹45.1 mg pantoprazole sodium sesquihydrate is equal to 40.0 mg pantoprazole free acid, while 22.55 mg of sodium pantoprazole sesquihydrate is equivalent to 20 mg of pantoprazole free acid.
²1.44 mg trisodium phosphate is equal to 3.34 mg trisodium phosphate 12-hdte.
³Purified water disappears in the manufacturing process.
⁴Eudragit L30-D55 ® is an aqueous dispersion, so water quantity disappears in the manufacturing process.

### EXAMPLE 3

### Dissolution Characteristics of Formulations of Pantoprazole Sodium Sesquihydrate

The amount of dissolved pantoprazole/pantoprazole sodium sesquihydrate can be determined conventionally using a UV absorption method and measured at 293 nm. Data is quantified by interpolation of the absorption results from the sample in a plot that shows a linear range of concentration versus absorbance.

Tablet (40 mg) from Example 2 and commercially available 40 mg pantoprazole tablets were tested for in vitro drug release in 900 mL of 0.1 N HCl for 2 hours. After 2 hours, the tablets were introduced in a phosphate buffered saline (pH=6.8) solution, and samples were taken at a regular interval basis. A USP-2 apparatus with paddle speed at 100 rpm was used for the study. The tablets showed gastroresistance with no dissolution of the pantoprazole when exposed to an HCl (pH=1.2) media over 2 hours.

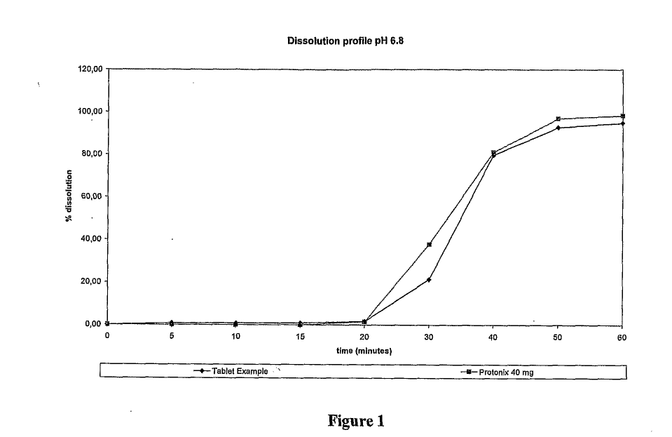

Table 3, below, illustrates the dissolution results in pH 6.8 media and FIG. 1 illustrates the same results graphically.

TABLE 3

| Time | Tablet Example 2 | Protonix ® |
|---|---|---|
| (minutes) | % Drug Release Profile | % Drug Release Profile |
| 0 | 0.00 | 0.00 |
| 5 | 0.71 | 0.00 |
| 10 | 0.77 | 0.00 |
| 15 | 0.88 | 0.00 |
| 20 | 1.56 | 1.42 |
| 30 | 21.48 | 37.96 |
| 40 | 79.72 | 81.01 |
| 50 | 92.82 | 96.96 |
| 60 | 94.91 | 98.34 |

### EXAMPLE 4

### Bioavailability Characteristics of Formulations of Pantoprazole Sodium Sesquihydrate

The bioavailability of pantoprazole sodium (40 mg) tablets prepared according to the invention was compared to the bioavailability of the marketed pantoprazole sodium 40 mg tablets (Ulcotenal® of Altana Pharma AG) in a single center, single-dose, open-label, randomized, two-treatment, two-period, two-sequence crossover in design, bioequivalence study under fasting conditions. The washout interval between study periods was one week.

The bioequivalence study included 30 healthy male and female volunteers. Venous blood samples to determine concentration of pantoprazole were then at baseline and at 1, 1.5, 2, 2.33, 2.66, 3, 3.33, 3.66, 4, 4.33, 4.66, 5, 6, 8, 10, 12 hour intervals. Plasma samples were analyzed to determine the concentration of pantoprazole. Pantoprazole was measured by reversed phase high performance liquid chromatography and detected by tandem mass spectrometry detection (LC-MS/MS).

The main analysis of this study focused on the kinetic parameters of pantoprazole (AUC, Cₘₐₓ), of which the most representative is AUCₗₐₛₜ. Individual analysis of variance (ANOVA) was performed on the In-transformed data of AUCₗₐₛₜ, AUC_{inf} and Cₘₐₓ. The Tₘₐₓ was also determined. For Tₘₐₓ, the analysis was based on the untransformed data using a non-parametric method (CI of the median of the differences, Wilcoxon test).

Tables 4 and 5 illustrate the results of the comparative bioavailability study in which formulation T (Test) is the formulation of Example 2 and formulation R (Reference) is the commercially available formulation from Altana.

TABLE 4
GLOBAL DESCRIPTIVE ANALYSIS OF PHARMACOKINETICS PARAMETERS OF
PANTOPRAZOLE (PAN-2004/008) (Units: Cₘₐₓ: ng/mL; AUC: ng · h/mL; Tₘₐₓ: h)

| Variable | Formulation | Mean | SD | Min | Median | Max | Geometric_Mean |
|---|---|---|---|---|---|---|---|
| Tₘₐₓ | R | 2.83 | 0.93 | 1.50 | 2.67 | 6.00 | 2.71 |
| Tₘₐₓ | T | 2.52 | 0.92 | 1.00 | 2.33 | 5.00 | 2.37 |
| Cₘₐₓ | R | 2596.33 | 974.53 | 225.96 | 2620.20 | 4457.74 | 2340.41 |
| Cₘₐₓ | T | 2525.31 | 844.56 | 1488.67 | 2261.21 | 4342.14 | 2400.30 |
| AUC_{INF} | R | 4833.29 | 2779.51 | 1988.32 | 3612.43 | 11114.11 | 4194.79 |
| AUC_{INF} | T | 4841.28 | 2385.22 | 2149.20 | 3641.72 | 9188.11 | 4318.72 |
| AUCₗₐₛₜ | R | 4729.73 | 2739.83 | 1503.33 | 3472.53 | 10909.66 | 4081.07 |
| AUCₗₐₛₜ | T | 4766.00 | 2346.24 | 2118.44 | 3578.96 | 9079.21 | 4250.38 |
| T_{1/2} | R | 1.24 | 0.58 | 0.53 | 1.11 | 3.44 | 1.14 |
| T_{1/2} | T | 1.20 | 0.41 | 0.62 | 1.06 | 2.17 | 1.14 |

TABLE 5

| | Ratio (% Ref) | CI 90 Lower Limit | CI 90 Upper Limit |
|---|---|---|---|
| Ln(Cₘₐₓ) | 102.56 | 85.85 | 122.52 |
| Ln(AUCₗₐₛₜ) | 104.15 | 93.39 | 116.15 |
| Ln(AUC_{INF}) | 102.95 | 93.56 | 113.29 |

According to the Guidance on bioavailability and bioequivalence (CPMP/EWP/QWP/1401/98), bioequivalence between formulations is established if the 90% confidence interval of the last-square means ratios of the test to reference products of in-transformed AUCₗₐₛₜ and Cₘₐₓ are within an acceptance range of 80 to 125%. As illustrated in Tables 4 and 5, the formulations of pantoprazole of the invention meet the bioequivalence criteria of the Guidance relative to the commercially available reference pantoprazole formulation.

Although the invention has been described and illustrated with a certain degree of particularity, it is understood that the present disclosure has been made only by way of example, and that numerous changes in the conditions and order of steps can be resorted to by those skilled in the art without departing from the spirit and scope of the invention.

What is claimed is:

1. A formulation of a pharmaceutically acceptable quantity of pantoprazole and/or its pharmaceutically acceptable salts comprising:
(i) a core comprising said pantoprazole and/or its pharmaceutically acceptable salts and at least one excipient material;
(ii) an inert intermediate layer surrounding said core, wherein said intermediate layer insulates said core; and
(iii) an outer layer surrounding said intermediate layer, wherein said outer layer is resistant to gastric juice.

2-3. (canceled)

4. The formulations of any of claim 1, wherein said pharmaceutically acceptable quantity of pantoprazole and/or its pharmaceutically acceptable salts is 100 mg or less when expressed as pantoprazole free acid.

5. The formulations of claim 1, wherein said pharmaceutically acceptable quantity of pantoprazole and/or its pharmaceutically acceptable salts is 40 mg or less when expressed as pantoprazole free acid.

6. The formulations of claim 1, wherein said pharmaceutically acceptable quantity of pantoprazole and/or its pharmaceutically acceptable salts is 20 mg or less when expressed as pantoprazole free acid.

7. The formulations of any of claim 1, wherein said core further comprises at least one excipient material.

8. The formulation of claim 7, wherein said pantoprazole and/or its pharmaceutically acceptable salts includes at least one of pantoprazole, pantoprazole sodium monohydrate, pantoprazole sodium sesquihydrate, pantoprazole magnesium and hydrates thereof and combinations thereof.

9. The formulation of claim 1, wherein said at least one excipient material is at least one of a binder, a filler, a disintegrant, a lubricant, a pH regulator and combinations thereof.

10. The formulation of claim 9, wherein said at least one excipient material is at least one of mannitol, spray dried mannitol, croscarmellose sodium, calcium stearate, trisodium phosphate or a hydrate thereof and combinations thereof.

11. The formulation of claim 9, wherein said pH regulator is at least one of trisodium phosphate, a hydrate thereof and combinations thereof.

12. The formulation of claim 9, wherein said mannitol is at least one of mannitol powder, spray dried mannitol and combinations thereof.

13. The formulation of claim 10, wherein said trisodium phosphate is used in an amount of approximately 0.8 mg of trisodium phosphate per 20 mg of said pantoprazole when expressed as free acid.

14. The formulation claim 1, wherein said intermediate layer comprises at least one of a polymer, a plasticizer and combinations thereof.

15. The formulation of claim 14, wherein said intermediate layer comprises at least one of hydroxypropylmethylcellulose, polyvinylpyrrolidone, propylene glycol and combinations thereof.

16. The formulation of claim 1, wherein said outer layer comprises at least one of a copolymer of methacrylate/acrylic acid, a plasticizer, a dye and combinations thereof.

17. The formulation of claim 16, wherein said outer layer comprises at least one of ethyl acrylate-methacrylic acid copolymer (1:1), triethyl citrate and combinations thereof.

18. The formulation of claim 1, wherein said core comprises pantoprazole sodium sesquihydrate, mannitol powder, trisodium phosphate or a hydrate thereof, croscarmellose sodium, spray dried mannitol and calcium stereate;
wherein said intermediate layer comprises hydroxypropylmethylcelulose, polyvinylpyrrolidone and propylene glycol; and
wherein said outer layer comprises ethyl acrylate-methacrylic acid copolymer (1:1), triethyl citrate, yellow ferric oxide A and titanium dioxide.

19-20. (canceled)

21. The formulation of claim 18, wherein said formulation is a 20 mg tablet of pantoprazole, said core comprising approximately 22.550 mg of pantoprazole sodium sesquihydrate, 39.675 mg of mannitol powder, 0.800 mg of trisodium phosphate monohydrate, 0.775 mg of croscarmellose sodium, 12.195 mg of spray dried mannitol and 1.505 mg of calcium stereate;
said intermediate layer comprising approximately 11.88 mg of hydroxypropylmethylcelulose, 0.24 mg of polyvinylpyrrolidone and 2.66 mg of propylene glycol; and
said outer layer comprising approximately 7.94 mg of ethyl acrylate-methacrylic acid:copolymer (1:1), 0.82 mg of triethyl citrate, 0.02 mg of yellow ferric oxide A, and 0.21 mg of titanium dioxide.

22. The formulation claim 18, wherein said formulation is a 40 mg tablet of pantoprazole, said core comprising approximately 45.1 mg of pantoprazole sodium sesquihydrate, 79.35 mg of mannitol powder, 1.6 mg of trisodium phosphate monohydrate, 1.55 mg of croscarmellose sodium, 24.39 mg of spray dried mannitol and 3.01 mg of calcium stereate;
said intermediate layer comprising approximately 19.00 mg of hydroxypropylmethylcelulose, 0.38 mg of polyvinylpyrrolidone and 4.25 mg of propylene glycol; and
said outer layer comprising approximately 14.13 mg of ethyl acrylate-methacrylic acid:copolymer (1:1), 1.45 mg of triethyl citrate, 0.03 mg of yellow ferric oxide A, and 0.34 mg of titanium dioxide.

23. The formulation claim 18, wherein said formulation is a 20 mg tablet of pantoprazole, said core comprising approximately 22.550 mg of pantoprazole sodium sesquihydrate, 39.775 mg of mannitol powder, 0.720 mg of trisodium phosphate (anhydrous), 0.775 mg of croscarmellose sodium, 12.195 mg of spray dried mannitol and 1.505 mg of calcium stereate;
said intermediate layer comprising approximately 11.88 mg of hydroxypropylmethylcelulose, 0.24 mg of polyvinylpyrrolidone and 2.66 mg of propylene glycol; and
said outer layer comprising approximately 7.94 mg of ethyl acrylate-methacrylic acid:copolymer (1:1), 0.82 mg of triethyl citrate, 0.02 mg of yellow ferric oxide A, and 0.21 mg of titanium dioxide.

24. The formulation claim 18, where said formulation is a 40 mg tablet of pantoprazole, said core comprising approximately 45.1 mg of pantoprazole sodium sesquihydrate, 79.51 mg of mannitol, 1.44 mg of trisodium phosphate (anhydrous), 1.55 mg of croscarmellose sodium, 24.39 mg of spray dried mannitol and 3.01 mg of calcium stereate;
said intermediate layer comprising approximately 19.00 mg of hydroxypropylmethylcelulose, 0.38 mg of polyvinylpyrrolidone and 4.25 mg of propylene glycol; and
said outer layer comprising approximately 14.13 mg of ethyl acrylate-methacrylic acid:copolymer (1:1), 1.45 mg of triethyl citrate, 0.03 mg of yellow ferric oxide A, and 0.34 mg of titanium dioxide.

25. A process for preparing the formulation of claim 1,
said process comprising a granulation step, an intermediate finishing step, a compression step, ad an insulating coating step and an enteric coating step.

26. The process of claim 25, wherein the at least one excipient material is mannitol powder.

27. The process of claim 25, wherein said granulation step comprises:
sieving and combining the desired quantities of pantoprazole and/or its pharmaceutically acceptable salts and the at least one excipient material;
granulating the obtained mixture with at least one of water and an aqueous solution of at least one excipient to obtain a granulate; and
drying the obtained granulate.

28. The process of claim 27, wherein the aqueous solution further comprises at least one alkaline reacting compound.

29. The process of claim 25, wherein said intermediate finishing step comprises:
mixing the product of said granulation step with spray dried mannitol in a container blender; and
combining a desired quantity of calcium stearate with the mixture of the product of said granulation step and the spray dried mannitol.

30. The process of claim 25, wherein said compression step comprises:
compressing the product of said intermediate finishing step to produce a core.

31. The process of claim 25, wherein said insulating step comprises:
preparing a solution of propylene glycol dissolved in deionized water;
preparing a mixture by adding polyvinylpyrrolidone and hydroxypropylmethylcelulose to the solution;
coating the product of said compression step with the mixture; and
allowing the mixture to dry.

32. The process of claim 25, wherein said enteric coating step comprises:
preparing a solution by mixing triethyl citrate, yellow ferric oxide A and titanium dioxide in deionized water;
adding the solution over an aqueous dispersion of ethyl acrylate-methacrylic acid:copolymer (1:1);
stirring the solution and the aqueous dispersion of ethyl acrylate-methacrylic acid:copolymer (1:1);
coating the solution and the aqueous dispersion of ethyl acrylate-methacrylic acid:copolymer (1:1) on the product of said insulating step; and
allowing the solution and the aqueous dispersion of ethyl acrylate-methacrylic acid:copolymer (1:1) to dry on the product of said insulating step.

33. A formulation of a pharmaceutically acceptable quantity of pantoprazole and/or its pharmaceutically acceptable salts comprising:
a core comprising an admixture of pantoprazole and/or its pharmaceutically acceptable salts with mannitol, an alkaline reacting compound, a tablet-disintegrating agent;
an inert intermediate layer surrounding and insulating said core; and
an outer layer surrounding said intermediate layer, said outer layer being resistant to gastric juice
wherein said mannitol is at least one of mannitol powder, spray dried mannitol and combinations thereof;
wherein said core is prepared by tableting a mixture of a granulate containing said pantoprazole and/or its pharmaceutically acceptable salts, said mannitol, said alkaline reacting compound and said tablet-disintegrating agent; and
wherein said alkaline reacting compound is trisodium phosphate and/or hydrates thereof.

34. The formulation of claim 33, wherein said core further comprises a lubricant.

35. The formulation of claim 34, wherein said lubricant agent is spray dried mannitol.
